(19) United States
(12) Patent Application Publication
(10) Pub. No.: US 2024/0093253 A1
(43) Pub. Date: Mar. 21, 2024

(54) SUSTAINABLE PRODUCTION OF CANNABINOIDS FROM SIMPLE PRECURSOR FEEDSTOCKS USING SACCHAROMYCES CEREVISIAE

(71) Applicant: National University of Singapore, Singapore (SG)

(72) Inventors: Kevin Jie Han Lim, Singapore (SG); Maybelle Darlene Kho Go, Singapore (SG); Wen Shan Yew, Singapore (SG)

(21) Appl. No.: 17/767,957

(22) PCT Filed: Oct. 12, 2020
(86) PCT No.: PCT/SG2020/050584

§ 371 Date: Apr. 11, 2022

Related U.S. Application Data
(60) Provisional application No. 62/914,058, filed on Oct. 11, 2019.

(51) Int. Cl. C12P 17/06; C12N 9/00; C12N 9/10; C12N 9/88; C12N 15/52; C12N 15/81

(52) U.S. Cl. CPC C12P 17/06; C12N 9/1029; C12N 9/1085; C12N 9/88; C12N 9/93; C12N 15/52; C12N 15/81; C12Y 203/01086; C12Y 203/01206; C12Y 205/01039; C12Y 404/01026; C12Y 602/01012; C12Y 602/01025; C12Y 602/0103; C12N 2800/102

(57) ABSTRACT

A recombinant cell of Saccharomyces cerevisiae that includes in its genome nucleic acids encoding cannabinoid biosynthetic pathway genes. A cannabinoid is produced by the recombinant cell in the presence of a cannabinoid precursor substrate and at least one of the cannabinoid biosynthetic pathway genes is from an organism other than Cannabis sativa, wherein the at least one of the cannabinoid biosynthetic pathway genes encodes a prenyltransferase. In an embodiment, the prenyltransferase is NphB from Streptomyces sp. having the amino acid sequence of any one of SEQ ID NOs: 8-11. Also disclosed is a method for producing a cannabinoid with the recombinant cell and the cannabinoid precursor substrate.

### BACKGROUND

Research and therapeutic use of phytocannabinoids, which are compounds originally isolated from the plant Cannabis. sativa, has been largely hindered due to legal and social issues. Some cannabinoids such as Δ⁹-tetrahydrocannabinol (THC), produced as a mixture with other cannabinoids in the plant, are found to be psychoactive. However, there are at least 113 known cannabinoids isolated from C. sativa (Aizpurua-Olaizola et al., J. Nat. Prod., 2016, 79(2):324-331), most of which are non-psychoactive and have distinct pharmacological properties.

The widespread expression of human cannabinoid receptors implies that these chemical compounds have an extensive effect on the human body. There are two main classes of receptors—CB₁ receptors are expressed mainly in the central nervous system, while CB₂ receptors are found predominantly in the peripheral immune system. In addition, studies have also uncovered expression of these receptors in various human tissues such as the heart, adrenal gland, lung, spleen, and tonsils (Galiègue et al., Eur. J. Biochem., 1995, 232(1):54-61). The association of cannabinoids with these receptors actuates signaling pathways that can potentially affect a wide range of biological systems in the human body.

Phytocannabinoids have been approved for use clinically by the US Food and Drug Administration as anti-emetic agents for patients undergoing intensive chemotherapy cancer treatment (THC and Dronabinol; Pertwee, Forsch. Komplementarmed., 1999, Suppl 3:12-15) and in the United Kingdom as a highly effective treatment for spasticity in patients with multiple sclerosis who are non-responsive to other medications (Flachenecker et al, Eur. Neurol., 2014, 71(5-6):271-279). Various studies have also shown the therapeutic potential of phytocannabinoids as anti-tumor agents (Velasco et al., Nat. Rev. Cancer, 2012, 12(6):436-444; Maria Pyszniak et al., Onco. Targets Therapy, 2016,.9:4323-4336) and anti-bacterial agents that may be effective against several strains of methicillin-resistant Staphylococcus aureus (MRSA) (Appendino et al., J. Nat. Prod., 2008, 71(8):1427-1430).

In light of these recent developments in cannabinoid research, the legal cannabis market is currently estimated to be worth USD7.7 billion internationally and this is expected to grow to around USD31.4 billion by 2021 (Zhang, Forbes, 2017).

Currently, most of the cannabinoids for research or therapeutic use are produced from C. sativa extracts. Legal and social implications associated with the illicit use of the plant extracts poses the greatest challenge faced by traditional cultivation due to the high concentrations of psychoactive components such as THC, produced as a mixture in C. sativa. There is limited control in the selectivity of cannabinoids that are produced in the cultivation of C. sativa. Furthermore, there is a huge gap in the market for C. sativa strains producing minor cannabinoids such as cannabidivarin (CBDV) and cannabicyclol (CBL) for research into their therapeutic potential.

The cultivation of these plants is not only expensive due to the need for highly energy-intensive processes to control environmental factors, it is also environmentally unsustainable. Research conducted in 2012 by the Energy & Resources group in the Lawrence Berkeley National Laboratory estimated that the cost of energy consumption from the indoor practice of cannabis cultivation in the US alone was around USD 6 billion annually. Further, greenhouse gas emissions from this practice to produce a kilogram of the final product is equivalent to that of 3 million cars (Mills, Energy Policy, 2012, 46:58-67).

There exists a need to find alternative means of cannabinoid production that circumvents the complications described above.

### SUMMARY

To meet the need set out, supra, a recombinant cell of Saccharomyces cerevisiae is provided that contains in its genome a plurality of nucleic acids each encoding a cannabinoid biosynthetic pathway gene such that a cannabinoid is produced by the recombinant cell in the presence of a cannabinoid precursor substrate. At least one of the cannabinoid biosynthetic pathway genes is not from Cannabis sativa.

Also disclosed is a method for producing a cannabinoid by contacting the recombinant cell with a cannabinoid precursor substrate and culturing the recombinant cell.

The details of one or more embodiments are set forth in the description and the examples below. Other features, objects, and advantages will be apparent from the detailed description, from the drawings, and also from the appended claims.

### BRIEF DESCRIPTION OF THE DRAWINGS

The invention description below refers to the accompanying drawings, of which:

FIG. 1 shows the biosynthetic pathway of cannabinoids. The polyketide pathway is colored in black and the isoprenoid pathway is colored in light gray. Modifications made to the pathway are colored in dark gray.

FIG. 2 shows results from the Transmembrane helices prediction webserver (TMHMM) for geranylpyrophosphate:olivetolate geranyltransferase showing 6 predicted transmembrane regions in its amino acid sequence.

FIG. 3A is a schematic representation of the YeastFab pathway assembly system showing the cloning procedures from assembly from biological parts (Level 0) of pathways (Level 2). Image taken from Guo et al., Nucleic Acids Res., 2015,. 43(13):e88.

FIG. 3B shows sequences of level 1 transcriptional units (POT) prefix and suffix for assembly to level 2 pathway.

FIG. 4 shows extracted ion count (left side) and mass spectrum (right side) of the LC-MS analysis of cannabinoids produced in S. cerevisiae: (first row) negative control construct 4ME expressing 4CL and MCS; (second row) construct 4MOO, expressing 4CL, MCS, OLS and OAC; and (last row) OLA standard.

FIG. 5 shows extracted ion count and mass spectrum of the LC-MS analysis of in vitro enzymatic assays: no enzyme negative control with GPP and OLA (first row), enzyme GOT with GPP and OLA (second row), enzyme NphB with GPP and OLA (third row), and CBGA standard (last row).

FIG. 6 shows a simplified mevalonate pathway overexpressing monoterpene precursors IPP, DMAPP and GPP together with geraniol biosynthesis in S. cerevisiae (Zhao et al., Appl. Microbiol. Biotechnol., 2016, 100(10):4561-4571). Adapted genes for producing GPP in a system of the invention are underlined.

FIG. 7 shows extracted ion count and mass spectrum of the LC-MS analysis of cannabinoids produced in S. cerevisiae: negative control is construct 4MOON pMKU+Empty pCKL expressing only the cannabinoid genes (first row), construct 4MOON pMKU+IHUE pCKL, expressing the cannabinoid genes on pMKU vector and the mevalonate genes on pCKL (second row), and CBGA standard (last row).

FIG. 8 shows the difference in carbon chain length between OLA and DVA (Groom et al., Acta Crystallogr. B Struct. Sci. Cryst. Eng. Mater, 2016, 72(Pt 2):171-179).

FIG. 9 shows production of OLA analogues using different starter units and Saccharomyces cerevisiae constructs.

FIG. 10 shows in column 3 the structures of novel olivetolic acid analogues produced in E. coli identified by extracted ion count LC-MS analysis. The E. coli expressed the biosynthetic pathways shown in column 1 and were incubated with the substrates shown in column 2. 4MOO=4-coumaryl-CoA ligase, malonyl-CoA synthethase (MCS), olivetol synthase (OLS), and olivetolic acid cyclase (OAC); BMOO=benzoate-CoA ligase, MCS, OLS, and OAC; CMOO=cinnamyl-CoA ligase, MCS, OLS, and OAC; and PMOO=phenylacetate-CoA ligase, MCS, OLS, and OAC.

FIG. 11 shows a proposed OLS reaction mechanism.

FIG. 12 shows protein structure of 3, 5-dioxodecanoyl-CoA intermediate (blue) docked into OLS structure (left panel) and 3, 5-dioxodecanoyl-CoA intermediate (blue) docked with superimposed malonyl-CoA unit (yellow).

### DETAILED DESCRIPTION

As summarized above, the recombinant cell provided contains in its genome a plurality of nucleic acids each encoding a cannabinoid biosynthetic pathway gene. In an exemplary recombinant cell, the cannabinoid biosynthetic pathway genes are from organisms other than Cannabis sativa.

Certain recombinant cells encompassed by the invention contain a nucleic acid that encodes a coenzyme A (CoA) ligase. The CoA ligase can be, but is not limited to, Nicotiana tabacum 4-coumaryl-CoA ligase (SEQ ID NO: 1), Rhodopseudomonas palstri benzoate-CoA ligase (SEQ ID NO: 2), and Streptomyces coelicolor phenylacetate-CoA ligase (SEQ ID NO: 3). The CoA ligase can have 70% or greater identity (e.g., 70%, 75%, 80%, 85%, 90%, 95%, and 99% or greater) to the above-mentioned sequences while retaining enzymatic activity.

Further, the recombinant cell can include nucleic acids encoding each of an a malonyl-CoA synthethase (MCS), an olivetol synthase (OLS), and an olivetolic acid cyclase (OAC). In particular recombinant cells, the MCS is replaced by an acyl-activating enzyme (AAE).

The MCS can have the amino acid sequence of SEQ ID NO: 5, the OLS can have the amino acid sequence of SEQ ID NO: 6, the OAC can have the amino acid sequence of SEQ ID NO: 7, and the AAE can have the amino acid sequence of SEQ ID NO: 4.

Alternatively, the MCS, OLS, OAC, and AAE can have an amino acid sequence having 70% or more identity to the amino acid sequence of their corresponding SEQ ID NOs.

The recombinant cell set forth above will produce a cannabinoid in the presence of a cannabinoid precursor substrate. The cannabinoid precursor substrate can be, e.g., butyric acid, valeric acid, hexanoic acid, heptanoic acid, and octanoic acid. Additional cannabinoid precursor substrates are shown in Table 1 below.

TABLE 1
Cannabinoid precursor substrates

| Substrate Category | Examples |
|---|---|
| cinnamic acids | cinnamic acid, 2-fluorocinnamic acid, 3- |
| | fluorocinnamic acid, 4-fluorocinnamic |
| | acid, α-fluorocinnamic acid, 2-chloro- |
| | cinnamic acid, 3-chlorocinnamic acid, |
| | 4-chlorocinnamic acid, 4- |
| | methylcinnamic acid, α-methylcinnamic |
| | acid, 2-hydroxy-cinnamic acid, 4- |
| | hydroxycinnamic acid, 2- |
| | methoxycinnamic acid, 4-methoxy- |
| | cinnamic acid, 2,4-dimethoxycinnamic |
| | acid, 3,4-dimethoxycinnamic acid, 3- |
| | chloro-4-methoxycinnamic acid, 4- |
| | hydroxy-3-methoxycinnamic acid, 4- |
| | hydroxy-3,5-dimethoxycinnamic acid, 4- |
| | nitrocinnamic acid |
| phenylpropanoic acids | 3-phenylpropanoic acid, 2-hydroxy-3- |
| | phenylpropanoic acid, 3-(2- |
| | bromophenyl) propanoic acid, 3-(2- |
| | methoxyphenyl) propanoic acid, 3-(3- |
| | methoxyphenyl) propanoic acid, 3-(4- |
| | methoxyphenyl) propanoic acid, 3-(3- |
| | chlorophenyl) propanoic acid, 3-(4- |
| | fluorophenyl) propanoic acid, 3-(3,4- |
| | dihydroxyphenyl) propanoic acid, 3- |
| | (3,5)-dichlorophenyl) propanoic acid, 3- |
| | (3,5)-difluorophenyl) propanoic acid, 2- |
| | hydroxy-3-(4-fluoro-phenyl)propanoic |
| | acid, 3-(3-chloro-4- |
| | methoxyphenyl)propanoic acid |
| benzoic acids | benzoic acid, 2-fluorobenzoic acid, 3- |
| | fluorobenzoic acid, 4-fluorobenzoic |
| | acid, 2-chlorobenzoic acid, 3- |
| | chlorobenzoic acid, 4-chlorobenzoic |
| | acid, 2-bromo-benzoic acid, 3- |
| | bromobenzoic acid, 4-bromobenzoic |
| | acid, 2-iodobenzoic acid, 3-iodobenzoic |
| | acid, 4-iodobenzoic acid, 2- |
| | aminobenzoic acid, 3-aminobenzoic |
| | acid, 4-aminobenzoic acid, 2-acetoxy |
| | benzoic acid, 2-hydroxy-3,5-diiodo |
| | benzoic acid, 2-hydroxybenzoic acid, |
| | 2-methoxybenzoic acid, 2- |
| | methylbenzoic acid, 2,3- |
| | dihydroxybenzoic acid, 2,4- |
| | dihydroxybenzoic acid, 2,5-dihydroxy |
| | benzoic acid, 2,6-difluorobenzoic acid, |
| | 2,6-dimethylbenzoic acid, 3,4- |
| | dimethoxy benzoic acid, 3,5- |
| | dihydroxybenzoic acid, 3,4,5- |
| | trihydroxybenzoic acid, 3,5-dihydroxy- |
| | 4-methoxybenzoic acid, 3,5- |
| | dimethylbenzoic acid, 4-amino-2- |
| | hydroxybenzoic acid |
| phenylacetic acids | phenylacetic acid, 2- |
| | hydroxyphenylacetic acid, 4- |
| | hydroxyphenylacetic acid, 4- |
| | methoxyphenylacetic acid, |
| | phenoxyacetic acid, 4- |
| | fluorophenoxyacetic acid, 2,4- |
| | dichlorophenoxyacetic acid, phenyl |
| | pyruvic acid |
| naphthalene, quinoline, and | l-naphthalenecarboxylic acid, 2- |
| pyridine carboxylic acids | naphthalenecarboxylic acid, 2-quinoline |
| | carboxylic acid, 3-quinolinecarboxylic |
| | acid, 4-quinolinecarboxylic acid, |
| | isoquinoline-1-carboxylic acid, 2- |
| | quinoxalinecarboxylic acid, pyridine-2- |
| | carboxylic acid, pyridine-3-carboxylic |
| | acid, pyridine-4-carboxylic acid, 2- |
| | chloropyridine-3-carboxylic acid, |
| | pyrazinecarboxylic acid |
| saturated aliphatic acids | acetic acid, propanoic acid, butanoic |
| | acid, pentanoic acid, hexanoic acid, |
| | heptanoic acid, octanoic acid, nonanoic |
| | acid, decanoic acid, pyruvic acid, |
| | acetoacetic acid, succinic acid, pimelic |
| | acid |
| unsaturated aliphatic acids | 2-propenoic acid, 2-methyl-2-propenoic |
| | acid, 2-butenoic acid, 3-butenoic acid, 2- |
| | methyl-2-butenoic acid, 3-methyl-2- |
| | butenoic acid, 2-pentenoic acid, 3- |
| | pentenoic acid, 4-pentenoic acid, 2- |
| | hexenoic acid, 3-hexenoic acid, 5- |
| | hexenoic acid, 3-methyl-4-pentenoic |
| | acid, shikimic acid |
| malonic acids | malonic acid, hydroxymalonic acid, |
| | methylmalonic acid, ethylmalonic acid, |
| | isopropylmalonic acid, n-butylmalonic |
| | acid, cyclopentylmalonic acid, |
| | allylmalonic acid, phenylmalonic acid, |
| | 3-thiophenemalonic acid, benzylmalonic |
| | acid |

As set forth in the SUMMARY section, a method for producing a cannabinoid is disclosed that uses the recombinant cell described above. The method is carried out by contacting the recombinant cell with a cannabinoid precursor substrate and culturing the recombinant cell.

In an exemplary method, the recombinant cell expresses a CoA ligase, a malonyl-CoA synthethase, an olivetol synthase, and an olivetolic acid cyclase, and the cannabinoid precursor substrate is butyric acid, valeric acid, hexanoic acid, heptanoic acid, or octanoic acid. Additional cannabinoid precursor substrates shown above in Table 1 can also be used in this method.

The cannabinoid biosynthetic pathway reconstituted synthetically in micro-organisms such as Saccharomyces cerevisiae is used for bio-production of major and minor cannabinoids. This method of bio-production facilitates the production of specific cannabinoids of interest, as compared to the mixture of cannabinoids produced by cultivation of C. sativa. Further, minor cannabinoids can be produced with high yield. In addition, enzymes in the cannabinoid biosynthetic pathway can be manipulated and expressed in micro-organisms to produce novel and unique cannabinoids that have therapeutic potential.

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever. All publications cited herein are incorporated by reference in their entirety.

### EXAMPLES

### Example 1: Modifications to Cannabinoid Biosynthetic Pathway

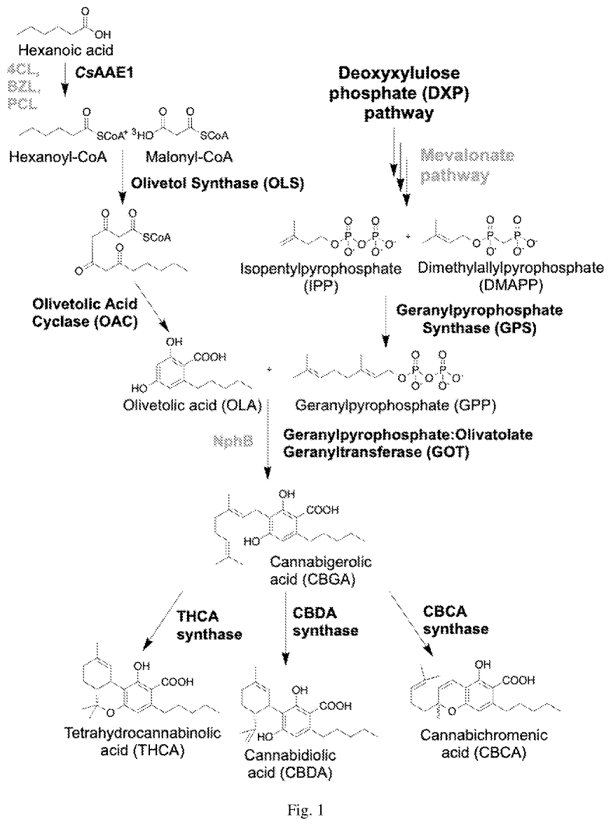

To reconstruct the cannabinoid biosynthetic pathway in micro-organisms, it is necessary to first understand the enzymes present in C. sativa that catalyze each step in the pathway. See FIG. 1.

The upstream biosynthetic pathway can be categorized into two functional parts, the polyketide pathway, producing olivetolic acid (OLA) as the final product and the isoprenoid pathway producing geranyl pyrophosphate (GPP).

### Polyketide OLA Production

The polyketide pathway (see FIG. 1, colored red) begins with the substrate hexanoic acid and malonyl-CoA. An acyl-activating enzyme named CsAAE1 (SEQ ID NO: 4) is responsible for the addition of the coenzyme-A moiety to hexanoic acid in the trichomes of C. sativa (Stout et al., Plant J., 2012, 71(3):353-365). A type III polyketide synthase named olivetol synthase (OLS; SEQ ID NO: 6) then catalyzes the formation of a tetraketide thioester (3, 5, 7-trioxododecanoyl-CoA) by utilizing one unit of hexanoyl-CoA and three units of malonyl-CoA (Taura et al., FEBS Lett, 2009, 583(12):2061-2066). Finally, olivetolic acid cyclase (OAC; SEQ ID NO: 7) catalyzes the C2-C7 aldol condensation cyclization step to produce OLA (Gagne et al., Proc. Natl. Acad. Sci. USA, 2012, 109(31):12811-12816).

### Isoprenoid GPP Production

In C. sativa, isoprenoids such as dimethylallyl-pyrophosphate (DMAPP) and isopentenyl-pyrophosphate (IPP) are produced through the 2-C-methyl-D-erythritol 4-phosphate (MEP) pathway (see FIG. 1, colored blue) (Van Bakel et al., Genome Biol., 2011, 12(10):R102). A putative geranyl pyrophosphate synthase then combines one unit of DMAPP with one unit of IPP to produce geranyl pyrophosphate (GPP).

Isoprenoids can also be produced in nature through the mevalonate pathway more commonly present in micro-organisms such as yeast and some bacteria (Buhaescu et al., Clin. Biochem., 2007, 40(9-10):575-584).

### End-Point Cannabinoids

Once OLA and GPP are produced, the aromatic prenyltransferase enzyme geranylpyrophosphate:olivetolate geranyltransferase (GOT) then catalyzes the production of the first cannabinoid, cannabigerolic acid (CBGA), by transferring the C10 prenyl group in GPP onto C₃ of olivetolic acid (Fellermeier et al., FEBS Lett., 1998, 427(2):283-285; see FIG. 1). The biosynthesis of different cannabinoids such as tetrahydrocannabinolic acid (THCA) and cannabidiolic acid (CBDA) can then be carried out by their respective synthases (i.e., THCA synthase and CBDA synthase) by differentially cyclizing the C10 carbon chain on CBGA (see FIG. 1, colored black) previously transferred from GPP.

### Modifications to Cannabinoid Biosynthetic Pathway

Modifications made to the cannabinoid biosynthetic pathway are illustrated in green in FIG. 1. A molecular toolkit was previously established to produce acyl-CoA thioesters with various functional groups using four different acyl-CoA ligases (Go et al., Biochemistry, 2012, 51(22):4568-4579). 4-coumaryl-CoA ligase (4CL) isolated from the plant Nicotiana tabacum (SEQ ID NO: 1), benzoate-CoA ligase (BZL) from the gram-negative bacteria Rhodopseudomonas palstri (SEQ ID NO: 2), and phenylacetate-CoA ligase (PCL) from the gram-positive bacteria Streptomyces coelicolor (SEQ ID NO: 3) were each determined to be substrate-promiscuous towards a diverse range of 70 carboxylic acids (see Table 1 above), including the native substrate of OLS, hexanoic acid. Further, malonyl-CoA synthetase (MCS) from the soil bacterium Rhizobium trifolii primes a number of malonic acids with a CoA moiety to serve as extender units for type III polyketide synthases such as OLS. Utilizing these well-characterized and substrate-promiscuous CoA-ligases in the pathway instead of CsAAE1 from C. sativa facilitates downstream production of diverse cannabinoids.

Each CoA-ligase is paired with malonyl-CoA synthethase (MCS), which is responsible for adding the CoA-moiety onto malonic acid.

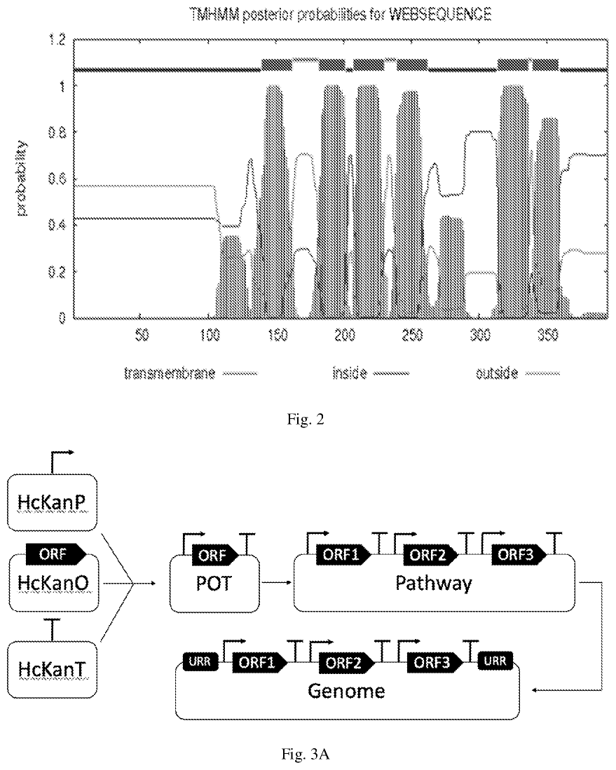

It is challenging to express the prenyltransferase from C. sativa, i.e., GOT, in a heterologous system. Indeed, this enzyme is predicted to be a membrane-associated protein with intrinsic trans-membrane regions, as shown by the bioinformatics prediction tool, TMHMM (Carvalho et al., FEMS Yeast Res., 2017, 17(4)). See FIG. 2.

A soluble prenyltransferase from Streptomyces sp., namely, NphB, was previously shown to be able to accept OLA as its prenyl-acceptor in bacterial systems such as E. coli (Kuzuyama et al., Nature, 2005, 435(7044):983-987; Yang et al., Biochemistry, 2012, 51(12):2606-2618). This enzyme was used as an alternative to GOT for heterologous expression of prenyl-transferases in the cannabinoid biosynthetic pathway.

### Example 2: Construct Assembly in Saccharomyces cerevisiae

The molecular cloning strategy for construct building in S. cerevisiae is a Golden-Gate assembly based suite of plasmids known as the YeastFab system (Guo et al.). The assembly system allows for the modular assembly of transcriptional units such as promoters and terminators to open reading frames (ORF), followed by the assembly of expression cassettes of up to six different transcriptional units.

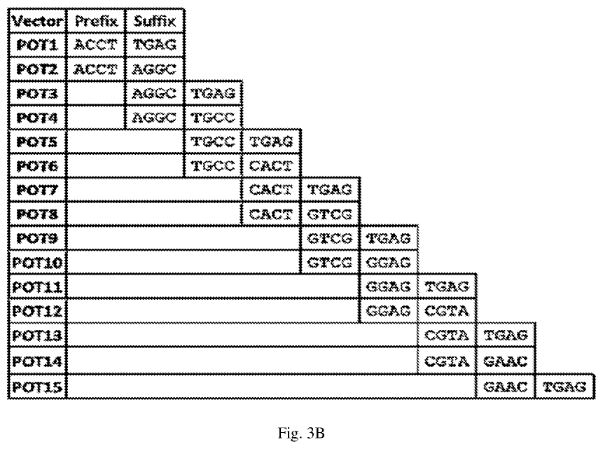

The suite of YeastFab plasmids has been expanded to allow for the assembly of up to eight transcriptional units together. The modular nature of assembly advantageously facilitates downstream optimization due to the relative ease in changing out transcriptional regulatory units such as promoters and terminators to regulate the expression of each gene differentially in the biosynthetic pathway. In this method of pathway assembly, Type IIS restriction enzymes (e.g., BsaI and BsmBI) cuts adjacent to the enzyme recognition site, allowing both restriction enzymes and DNA ligase to work in a one-pot digestion-ligation reaction. See FIG. 3A.

Standard biological parts such as promoters, open reading frames and terminators are individually cloned into individual plasmids (Level 0). After the first round of one-pot digestion-ligation reaction, they are assembled into individual transcriptional units called POTs (Level 1). Each POT is designed to then assemble with another POT in the following reaction to assemble up to eight transcription units together into a pathway (Level 2) through prefix and suffix sequences that aligns together as depicted in FIG. 3B.

A library of yeast constitutive promoters was previously characterized according to their relative strengths. A set of promoters and terminators with various strength were chosen to express each enzyme in the biosynthetic pathway, reflecting their relative stoichometries and toxicities to the expression host. Different promoters and terminators were assigned for each ORF (see Table 2 below) to prevent homologous recombination that occurs frequently in yeast between homologous sequences that are close to each other (Orr-Weaver et al., Proc. Natl. Acad. Sci. USA, 1981, 78(10):6354-6358).

TABLE 2
Promoters and terminators chosen for each ORF. (A) Pathway
assembly to reach OLA. (B) Pathway assembly to reach CBGA.
(C) Pathway assembly to reach end-point cannabinoid.

| | | | |
|---|---|---|---|
| POT | ORF | Promoter (Strength) | Terminator |

A: Construct to OLA

| | | | |
|---|---|---|---|
| 2 | CoA Lig | RPL3p (Strong) | APL2t |
| 4 | MCS | PGK1p (Strong) | ATG10t |
| 6 | OLS | TDH3p (Strong) | LSC2t |
| 7 | OAC | TEF2p (Strong) | BUD32t |

B: Construct to CBGA

| | | | |
|---|---|---|---|
| 2 | CoA Lig | RPL3p (Strong) | APL2t |
| 4 | MCS | PGK1p (Strong) | ATG10t |
| 6 | OLS | TDH3p (Strong) | LSC2t |
| 8 | OAC | TEF2p (Strong) | BUD32t |
| 10 | Erg20pWW/ | ENO2p (Strong) | EFM1t |
| | AgGPPase/ | | |
| | ChiGPPase | | |
| 11 | GOT/NphB | YEF3p (Medium)/ | CBR1t/EBS1t |
| | | TPI1p (Strong) | |

C: Construct to end-point cannabinoid

| | | | |
|---|---|---|---|
| 2 | CoA Lig | RPL3p (Strong) | APL2t |
| 4 | MCS | PGK1p (Strong) | ATG10t |
| 6 | OLS | TDH3p (Strong) | LSC2t |
| 8 | OAC | TEF2p (Strong) | BUD32t |
| 10 | Erg20pWW/ | ENO2p (Strong) | EFM1t |
| | AgGPPase/ | | |
| | ChiGPPase | | |
| 12 | GOT/NphB | YEF3p (Medium)/ | CBR1t/EBS1t |
| | | TPI1p (Strong) | |
| 13 | THCAS/CBDAs/ | SSA1p (Medium) | ENT2t |
| | CBCAs | | |

After assembly of the transcriptional units with the assigned promoters and terminators, they were assembled into a pathway with multiple genes in a single plasmid. In yeast, farnesyl pyrophosphate (FPP), which is a C15 version of GPP by adding an additional IPP to GPP, is endogenously produced by the enzyme Erg20p. Previous studies have shown that expressing a double mutant of the enzyme, i.e., Erg20pWW (F96W-N127W), increases endogenous GPP levels (Ignea et al., ACS Synth. Biol., 2014, 3(5):298-306). This double mutant, together with the geranyl diphosphate synthase (Ag-GPPase) from Abies grandis (Burke et al., Arch. Biochem. Biophys., 2002, 405(1):130-136) and a chimeric GPP synthase (Chi-GPPase), were used to produce GPP in the construct to feed the reaction forward.

### Example 3: Cannabinoid Bioproduction

After assembly of expression genes into their intended plasmids, they were then transformed into a single organism (S. cerevisiae) for bioproduction of the intended product. The yeast strain BY4741, which is auxotrophic for the amino acids methionine, leucine, histidine and uracil (Brachmann et al., Yeast, 1998, 14(2):115-132), was used for bio-production of cannabinoids. A step-wise approach can be taken as precautionary measure to ensure that yeast transformed with each plasmid subsequently expresses a functional enzyme. The product of each step (e.g., OLA, CBGA, and CBDA) was then detected using liquid chromatography—mass spectrometry (LC-MS) before moving on to the next step in the biosynthesis pathway. S. cerevisiae cultures were grown to stationary phase to express the enzymes.

Substrates, i.e., hexanoic acid and malonic acid, were added to the cultures, which were then incubated overnight at 25° C. Cultures were spun down to separate the pellets from the supernatant media. Both the cell pellets and supernatant media were checked for the presence of the intended product by LC-MS.

To prepare samples for LC-MS analysis, the supernatant media was acidified to pH<2.0 before extracting it three times with ethyl acetate. The extracted ethyl acetate was dried using a rotary vacuum concentrator, resuspended in methanol, and analysed by LC-MS. The cell pellets were also checked for presence of the intended product by firstly lysing the cells via glass-bead physical shearing or by chemical lysis. Consequently, the lysate underwent similar steps of acidification, organic extraction and LC-MS analysis.

### Example 4: Biosynthesis of Novel Cannabinoids

Diversification of the cannabinoid library can be carried out to produce novel and unnatural cannabinoids by three different approaches, informed by computational analysis.

First, the traditional approach of precursor-directed combinatorial biosynthesis involving the utilization of different substrates fed to the constructs will facilitate the exploration of substrate-promiscuity profiles of the enzymes in the pathway. Unique and novel products formed at each step then serve as new substrates for the next step in the pathway, hence increasing the diversity of products produced. Each enzyme has an innate specificity to its substrates that it can accept due to steric constraints and interactions in its binding pocket. Computational approaches such as in silico docking allows the screening of large libraries of small-molecule compounds in a high-throughput manner to identify substrates with a favorable binding affinity that is then tested experimentally.

Second, in silico docking of a substrate to the enzyme structure will clearly elucidate the spatial constraints and the interactions of the substrate in the microenvironment of the active site. This information is then used to direct protein engineering. It is vital to understand the key interactions of the substrate to the binding pocket of the enzyme in order to rationally design specific mutations that may increase the diversity of substrates that each enzyme may accept.

Finally, there is a limit to the degree of protein engineering that can be applied to each enzyme as mutations thus introduced may unintentionally change the architecture of the entire protein structure and result in protein instability and inactivity. Orthologues from different organisms of the enzymes carrying out each biosynthetic step can be identified and incorporated into the pathway. The Enzyme Similarity tool from the Enzyme-Function Initiative (EFI-EST) is a bioinformatics web server that aids in the identification of orthologues of a protein query sequence (Gerlt et al., Biochim. Biophys. Acta, 2015, 1854(8):1019-1037). The web tool uses the UniProt database to identify amino acid sequences in nature that are related to the query sequence in terms of function and reactions catalyzed. It facilitates the visualization of their relationships by generating a sequence similarity network that identifies proteins from diverse families which may have very different substrate-specificity profiles. The orthologues thus identified, having an entirely different set of substrates that it can act on, can serve as suitable replacements for particular steps in the cannabinoid biosynthesis pathway and consequently produce unique and novel cannabinoid products.

### Example 5: Cannabinoid Bio-Production in S. cerevisiae

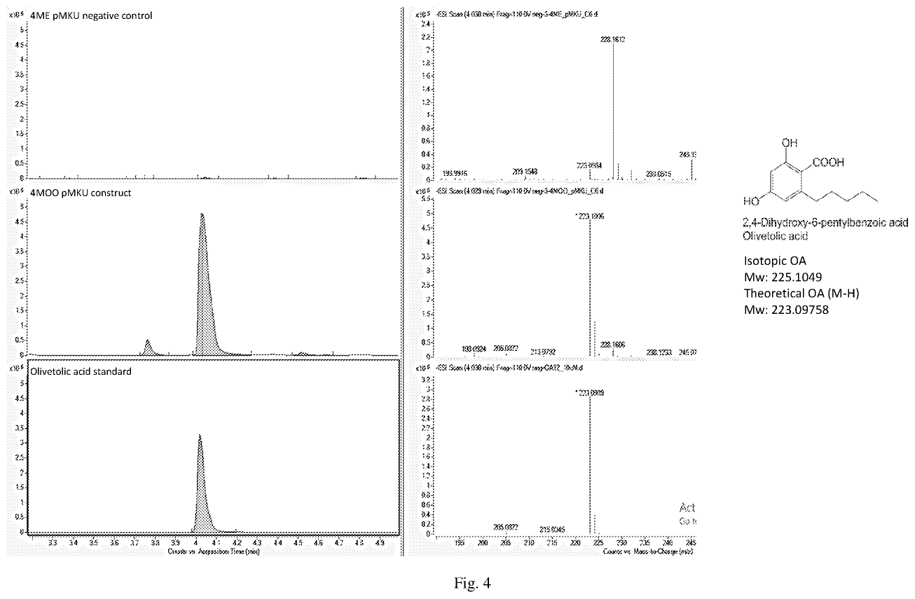

Transcriptional units were assembled into POTs as described above in Example 2. The plasmids were inserted into the level 2 assembly plasmids, pCKU and pMKU, which contain, respectively, the yeast low or high copy origins of replication CEN and 2 μ, as well as a URA3 selection marker. The assembled plasmids were transformed into individual strains of S. cerevisiae BY4741 using a previously described lithium acetate method (Gietz et al., Nat. Protoc., 2007, 2(1):35-37). These strains were grown overnight before substrates hexanoic acid and malonic acid were added to the cultures. After an overnight incubation at 25° C. with the substrates, cultures were harvested and growth media from each culture was extracted with ethyl acetate. The results are shown in FIG. 4.

Culture supernatants of yeast expressing four enzymes in the cannabinoid pathway, i.e., CoA ligase, MCS, OLS, and OAC, demonstrated a peak eluting at ˜4 min. having a m/z value of 223.100 (ppm error of 11.7), corresponding to that of an OLA. See FIG. 4, top and bottom rows. This peak was not seen in negative control supernatants from yeast lacking the polyketide synthase and polyketide cyclase, OLS and OAC See FIG. 4, top row.

The next step in the pathway produces CBGA. To do so, the first four genes mentioned above that produce OLA were assembled together with a GPP synthase enzyme (Erg20pWW, AgGPPase, or ChiGPPase) and a prenyltransferase, i.e., GOT or NphB, into the level 2 assembly plasmid pCKU and pMKU. After assembly of the transcriptional units and transformation into the host yeast strains, culture supernatants were analyzed by LC-MS as described above. A CBGA product was not observed in all of the constructs assembled, intracellularly or extracellularly. Not to be bound by theory, it is possible that the lack of CBGA in some cultures resulted from poor enzyme expression of the prenyltransferases (GOT or NphB) or a lack of substrate (OLA or GPP) available for the reaction.

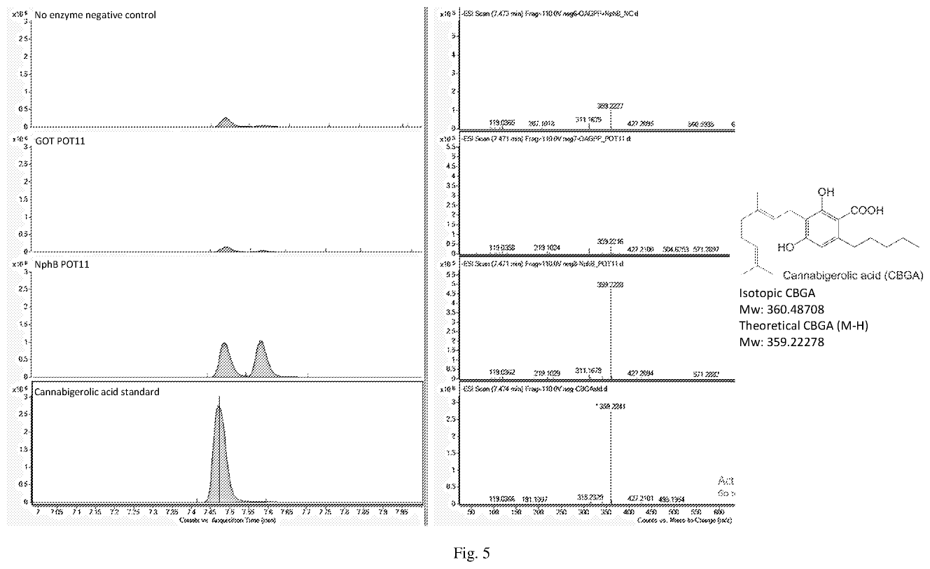

To test the activity of the prenyltransferase enzyme, level 1 POT plasmids expressing the transcriptional units of GOT and NphB prenyltransferase were separately transformed into BY4741 cells. The cultures were grown and lysed at stationary phase as described above. An aliquot of the lysate was then added separately into an in vitro enzymatic assay set up with substrates OLA and GPP. A negative control was also set up by replacing the lysate with water. After incubation at 30° C., acidification and organic extraction was done before LC-MS detection. The results are shown in FIG. 5.

A small peak corresponding to CBGA was observed in the negative control (without enzymes added) and the lysate containing GOT. See FIG. 5, first and second rows. This data suggests that the prenylation of the geranyl moiety onto OLA happens as a spontaneous process without any enzymatic catalysis and small amounts will be observed when OLA and GPP are present in a mixture. GOT expressed in S. cerevisiae BY4741 was determined to be inactive as the peak area corresponding to CBGA was similar to that of the negative control.

NphB-containing lysate, on the other hand, showed a peak area for CBGA larger than to that of the negative control, indicating that the NphB was active. See FIG. 5, third row. A secondary peak eluted 0.1 min. later was also observed in the sample catalysed by NphB with the same m/z value as CBGA. This peak was previously reported to be an unspecific product of OLA prenylated at a different site from CBGA, namely, 2-O-geranyl olivetolic acid (Zirpel et al., J. Biotechnol., 2017, 259:204-212). As NphB was shown to function when expressed in yeast strain BY4741, the poor production of CBGA must lie with the supply of substrates OLA and GPP in the constructs. Not to be bound by theory, it is possible that insufficient yields of GPP was the most likely cause of the low CBGA yield, as peaks corresponding to OLA were detected in all the constructs.

Methods for overexpressing monoterpenes derived from GPP in S. cerevisiae are well known in the art. There are an abundance of papers published describing work done in achieving improved monoterpene production in yeast due to their high commercial value. One approach to improve GPP yield is to reduce the metabolic burdens borne by the yeast host. It has been reported that, by overexpressing four genes involved in the mevalonate pathway and the respective monoterpene synthase, geraniol synthase, a seven-fold increase in geraniol yield was achieved, compared to previously engineered yeast strains (Zhao et al.).

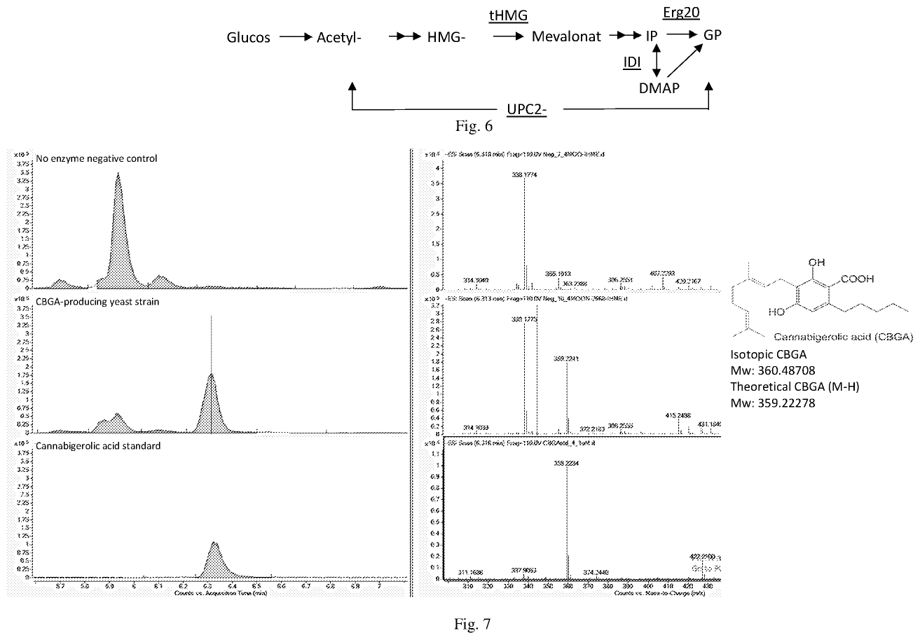

Referring to FIG. 6, the isoprenoid diphosphate isomerase (IDI1) catalyzes the isomerization between DMAPP and IPP. This helps to improve the ratio of precursors favored for GPP production (Liu et al., J. Biotechnol., 2013, 168(4):446-451). HMG-CoA was identified as a key rate-limiting step in the mevalonate pathway and overexpressing a truncated version of the gene, tHMG1, increases mevalonate supply for the pathway (Asadollahi et al., Biotechnol. Bioeng., 2010, 106(1):86-96; Scalcinati et al., Metab Eng, 2012. 14(2): p. 91-103). UPC2 is a transcription factor involved in the regulation of sterol biosynthesis and overexpressing a mutant, UPC2-1 enhances sterol uptake aerobically and consequently mevalonate production (Davies et al., Mol. Cell. Biol., 2005, 25(16):7375-7385).

The IDI1, tHMG1, and UPC2-1 genes were assembled together with the previously described GPP synthase, Erg20pWW, using the YeastFab system as described above into level 1 POTs and subsequently into a level 2 assembly plasmid (pCKL/pMKL) expressing auxotrophic marker LEU2. This allows the expression of genes for cannabinoids and isoprenoids to be maintained on separate plasmids in order to reduce the length of each plasmids assembled and transformed into the yeast strains.

A series of medium to strong promoters and terminators were individually assigned to overexpress the four genes for GPP production maintained on the pCKL/pMKL plasmid using the leucine auxotrophic marker (LEU2), while a different set of promoters and terminators were assigned to enzymes in the cannabinoid genes, maintained on a separate plasmid, pCKU/pMKU. See Table 3 below.

TABLE 3
Promoters and terminators chosen for (A) ORFs from the mevalonate pathway maintained
on pCKL/pMKL and (B) cannabinoid enzymes maintained on pCKU/pMKU.

| A: Construct to GPP | | | | B: Construct to CBGA | | | |
|---|---|---|---|---|---|---|---|
| | | Promoter | | | | Promoter | |
| POT | ORF | (Strength) | Terminator | POT | ORF | (Strength) | Terminator |
| 2 | IDI1 | GPM1 (Medium- | BNA4t | 2 | CoA Lig | RPL3p (Strong) | APL2t |
| | | Strong) | | | | | |
| 4 | tHMG1 | ACT1 (Medium- | PRX1t | 4 | MCS | PGK1p (Strong) | ATG10t |
| | | Strong) | | | | | |
| 6 | UPC2-1 | TDH2p (Medium) | YPT31t | 6 | OLS | TDH3p (Strong) | LSC2t |
| 7 | Erg20pWW | ENO2p (Strong) | EFM1t | 8 | OAC | TEF2p (Strong) | BUD32t |
| | | | | 9 | GOT/NphB | YEF3p | CBR1t/EBS1t |
| | | | | | | (Medium)/ | |
| | | | | | | TPI1p (Strong) | |

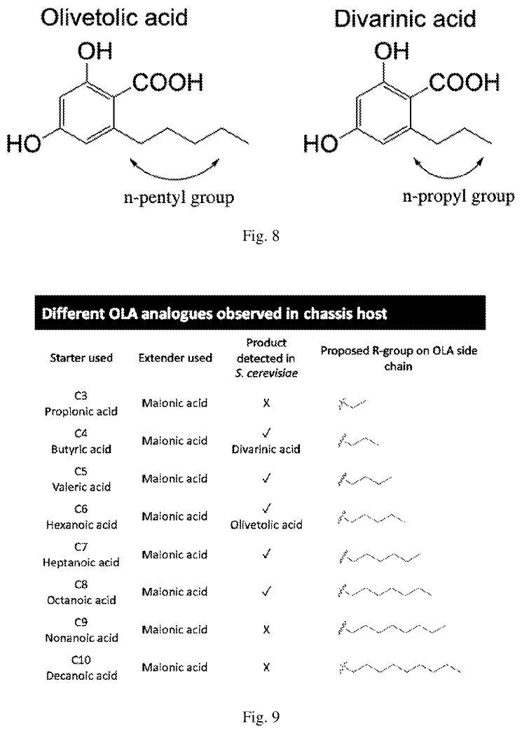

After assembly of both cannabinoid (pCKU/pMKU) and mevalonate genes was achieved, both plasmids were then transformed into the yeast strain BY4741 and grown in media lacking both uracil and leucine to select for transformants. The same bio-production procedure was carried out with constructs expressing both plasmids. Trace amounts of CBGA was detected intracellularly after conducting organic extraction of the lysed cell pellets. The results are shown in FIG. 7. Peaks with an elution time of ˜6.7 mins and an m/z value of 359.2223 (ppm error of −1.34) correspond to that of the CBGA standard.

To further increase yields of CBGA produced by the pathway expressed in yeast, computational protein engineering was conducted on wild-type NphB to improve its selectivity in producing CBGA instead of the side product, 2-O-geranyl olivetolic acid. Four mutants, i.e., V49W/Y288A (SEQ ID NO: 8), V49W/Y288P (SEQ ID NO: 9), V49W/Y288A/Q295F (SEQ ID NO: 10), and V49W/Y288P/Q295F (SEQ ID NO: 11) were generated that are highly specific in prenylating olivetolic acid using the geranyl moiety from GPP at the correct site to produce CBGA. These 5 mutants, which showed a significant increase in CBGA yield while producing no side products at all, were subsequently integrated into the pathway and CBGA titers improved significantly in vivo.

### Example 6: Diversification of Cannabinoids

Branch-points in the cannabinoid biosynthetic pathway can be exploited to produce novel cannabinoid compounds. The promiscuous nature of the acyl-CoA ligase and polyketide synthase, i.e., OLS, used in the above constructs allows for the use of different short-medium chain fatty acids (C3-C10) as substrates instead of hexanoic acid (C6) to produce analogues of olivetolic acid. An example in C. sativa is the production of the cannabinoids tetrahydrocannabivarin (THCV) and cannabidivarinic acid (CBDVA), which are both produced by the same enzymes in the pathway. These cannabinoids, produced in minor quantities, use the polyketide precursor divarinic acid (DVA) instead of OLA. DVA, which has a C3 chain-length on C₂ of the aromatic ring instead of a C5 chain in OLA (see FIG. 8) is also produced by CsAAE1 enzyme in C. sativa using butyric acid (C4) as a starter unit instead of hexanoic acid (C6).

OLS and OAC were tested for substrate-promiscuity by using different starter units (C3-C10) instead of hexanoic acid (C6). The results are shown in FIG. 9.

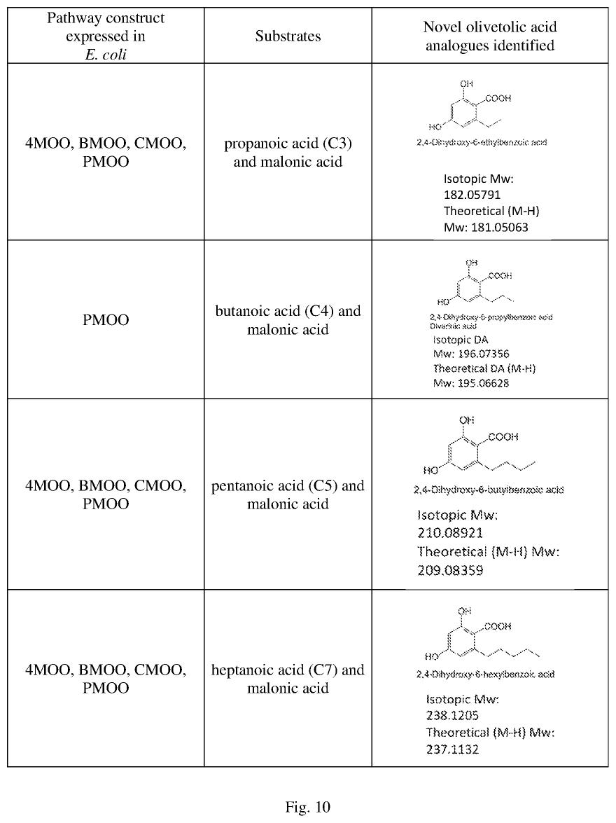

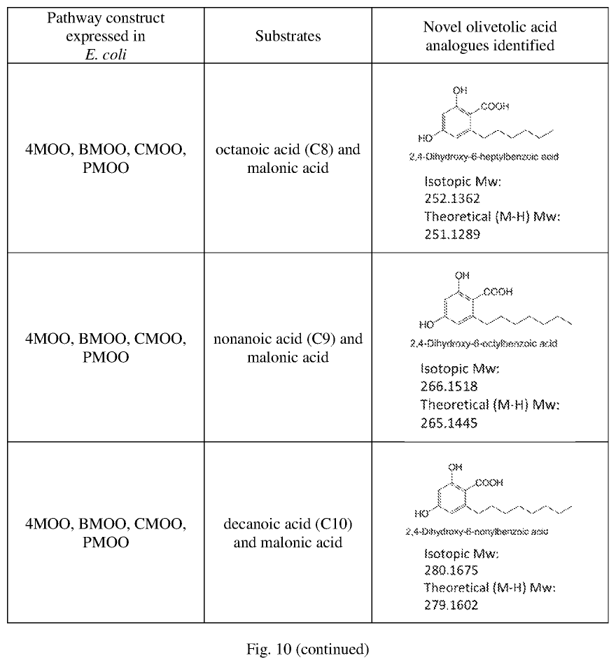

Certain OLA analogues, i.e., (C3, C9, C10), were not produced in S. cerevisiae but were produced using the same biosynthetic pathway constructs in E. coli. See FIG. 10. This could be due to the presence of an endogenous acyl-CoA ligase present in E. coli and not in S. cerevisiae that is able to act on substrates such as propionic acid, nonanoic acid, and decanoic acid. Such an endogenous ligase can provide a primed acyl-CoA thioester as a substrate for the polyketide synthase OLS to act on.

### Example 7: Further Diversification of Cannabinoid Production

The traditional method of precursor-directed combinatorial biosynthesis involves laborious screening of large libraries of substrates experimentally to detect if a new product was formed. In order to shorten the time and resources taken to identify favorable substrates, computational methods such as docking are employed as a prediction model to identify substrates that are able to fit into the binding pocket of the enzyme and that have favorable binding affinity to the active site. This facilitates screening of thousands of substrate libraries in a high-throughput manner to produce a ranked list of the most favorable substrates for experimental testing.

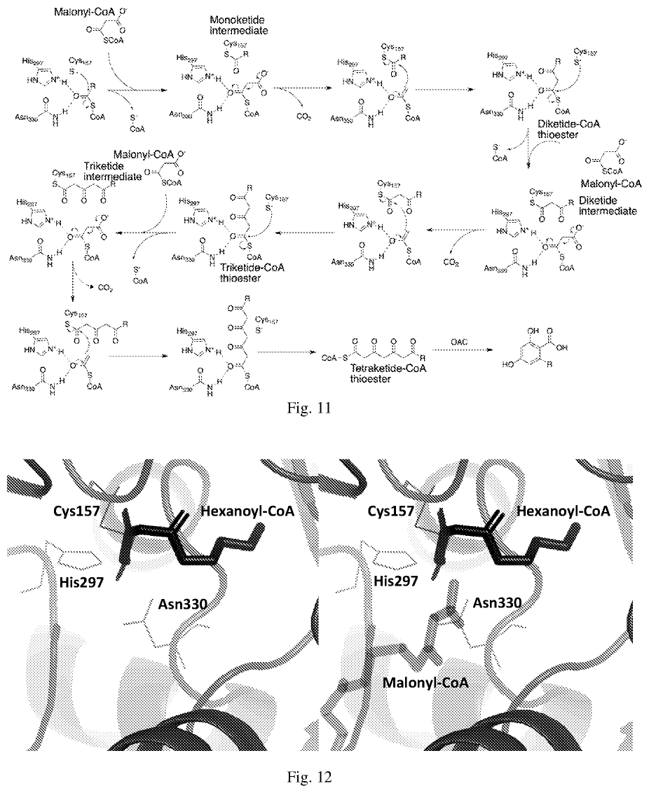

Starting with a 2.2 Å resolution structure of OLS, the native substrates of this enzyme, hexanoyl-CoA and malonyl-CoA were first docked to serve as a positive control. OLS uses two substrates with different R-groups, shuttled by a CoA moiety into the active site; a hexanoyl-CoA starter unit and three malonyl-CoA extender units that iteratively extends the starter unit by attaching acetyl-ketone groups onto the hexanoyl-intermediate attached to Cys157 of OLS as illustrated in FIGS. 11 and 12.

In order to identify different starter units that can perform the same three elongation steps using an extender unit such as malonyl-CoA, the last intermediate state that is still covalently attached to OLS, i.e., a triketide intermediate, 3, 5-dioxo decanoyl-CoA (after extension with two units of malonyl-CoA), was docked using GOLD (Groom et al.; see FIG. 12, left panel). This allows for delineation of the spatial constraints of accommodating the reaction mechanism of performing the multiple elongation steps. If the binding pocket is able to fit the last covalently attached intermediate state of the corresponding starter unit, it should be able to perform the first few rounds of elongation.

The last malonyl-CoA unit was then superimposed in using the crystallized malonyl-CoA ligand complexed with a homologous protein, chalcone synthase (CHS2) from the legume Medicago sativa (PDB code: 1CML) (Ferrer et al., Nat. Struct. Biol., 1999, 6(8):775-784). The superimposed malonyl-CoA ligand (yellow ligand in FIG. 12, right panel) was energy-minimized with the docked 3, 5-dioxodecanoyl-CoA intermediate (blue ligand in FIG. 12) in the OLS structure using the protein localization optimization program (PLOP) (Jacobson et al., J. Phys. Chem. B, 2002, 106(44):11673-11680; Jacobson et al., Proteins, 2004, 55(2):351-367; Zhao et al., Proteins, 2011, 79(10):2920-2935; Zhu et al., Proteins, 2006, 65(2):438-452; Zhu et al., J. Chem. Theory Comput., 2007, 3(6):2108-2119.). This last superimposition step identifies starter units that may have a bulkier R-group and result in steric clashes in the OLS binding pocket with the last malonyl-CoA ligand and thereby preventing the last extension step. These candidates are subsequently eliminated. As observed from the positive control in FIG. 12 left and right panels, the position of both the docked 3, 5-dioxodecanoyl-CoA intermediate from the hexanoyl-CoA starter unit and the energy-minimized malonyl-CoA primes the two substrates together for the last extension step.

As mentioned above a library of 70 acyl-CoA starter units (Go et al.) and 12 different acyl-CoA extender units (Go et al., ACS Catalysis, 2015, 5(7):4033-4042) is available. Collectively, this equates to a total of 840 combinations of starter and extender units that can serve as substrates for type III polyketide synthases such as OLS. These are used as the substrate library to be docked into the OLS structure. The outputs of the docking results are ranked accordingly and compared with experimental results to check for accuracy of the prediction model. This experimental testing of substrate promiscuity of OLS will produce olivetolic acid analogues with a varied chain length or R-group that can then be used to produce cannabinoids with a varied R-group on C₂ of the olivetolate-cannabinoid scaffold (see FIG. 8).

### Example 8: Protein Engineering

Protein engineering is a long established field of engineering enzymes to specific desired characteristics. Yet, without a good selection method, the vast amount of sequence space requres a prediction model in selecting mutants to display product diversity.

As mentioned above, NphB mutants were designed with a high selectivity for producing CBGA without any side product. Different NphB mutants were designed such that this prenyltransferase accepts different substrates and thus produces a different desired cannabinoid product.

The last cyclization step in the cannabinoid biosynthetic pathway cyclizes the C10 chain previously transferred from GPP (see FIG. 1). CBGA analogues are produced with a longer prenyl chain length that will serve as a precursor to more diverse structures due to the longer chain length available. A C15 chain length analogue of GPP, farnesyl pyrophosphate (FPP) and a C20 chain length analogue, geranylgeranyl pyrophosphate (GGPP) are used as suitable replacements for GPP to produce CBGA analogues with a C15 or C20 prenyl chain. Using the same computational protein engineering approach, specific mutations on the NphB enzyme can be identified that afford a better affinity for the longer FPP and GPP. These mutations can be tested to subsequently produce CBGA analogues.

An orthologue library of cannabinoid synthases are screened against CBGA-analogue producing strains to test for production of varied cannabinoid chemical structures that are subsequently tested for bioactivity.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims. What is claimed is:

1. A recombinant cell of Saccharomyces cerevisiae, comprising in its genome a plurality of nucleic acids each encoding a cannabinoid biosynthetic pathway gene, wherein a cannabinoid is produced by the recombinant cell in the presence of a cannabinoid precursor substrate and at least one of the cannabinoid biosynthetic pathway genes is from an organism other than Cannabis sativa.

2. The recombinant cell of claim 1, wherein one of the plurality of nucleic acids encodes a prenyltransferase.

3. The recombinant cell of claim 2, wherein the prenyltransferase is NphB from Streptomyces sp.

4. The recombinant cell of claim 2, wherein the prenyltransferase has the amino acid sequence of any one of SEQ ID NOs: 8-11 or an amino acid sequence at least 70% identical to SEQ ID NOs: 8-11 that has prenyltransferase activity.

5. The recombinant cell of claim 1, wherein one of the plurality of nucleic acids encodes a coenzyme A (CoA) ligase selected from the group consisting of Nicotiana tabacum 4-coumaryl-CoA ligase, Rhodopseudomonas palstri benzoate-CoA ligase, and Streptomyces coelicolor phenylacetate-CoA ligase.

6. The recombinant cell of claim 5, wherein the CoA ligase has the amino acid sequence of any one of SEQ ID NOs: 1-3 or an amino acid sequence at least 70% identical to SEQ ID NOs: 1-3 and having CoA ligase activity.

7. The recombinant cell of claim 1, wherein one of the plurality of nucleic acids encodes a malonyl-CoA synthethase (MCS), an olivetol synthase (OLS), and an olivetolic acid cyclase (OAC).

8. The recombinant cell of claim 7, wherein the MCS has the amino acid sequence of SEQ ID NO: 5 or an amino acid sequence 70% identical to SEQ ID NO: 5 having MCS activity.

9. The recombinant cell of claim 1, wherein the cannabinoid precursor substrate is selected from the cannabinoid precursor substrates listed in Table 1.

10. The recombinant cell of claim 9, wherein the cannabinoid precursor substrate is butyric acid, valeric acid, hexanoic acid, heptanoic acid, or octanoic acid.

11. A method for producing a cannabinoid, the method comprising contacting the recombinant cell of claim 1 with a cannabinoid precursor substrate and culturing the recombinant cell.

12. The method of claim 11, wherein the cannabinoid precursor substrate is selected from the cannabinoid precursor substrates listed in Table 1.

13. The method of claim 12, wherein the cannabinoid precursor substrate is butyric acid, valeric acid, hexanoic acid, heptanoic acid, or octanoic acid.

14. The method of claim 11, wherein one of the plurality of nucleic acids encodes a prenyltransferase.

15. The method of claim 14, wherein the prenyltransferase is NphB from Streptomyces sp.

16. The method of of claim 14, wherein the prenyltransferase has the amino acid sequence of any one of SEQ ID NOs: 8-11 or an amino acid sequence at least 70% identical to SEQ ID NOs: 8-11 that has prenyltransferase activity.

17. The method of claim 11, wherein one of the plurality of nucleic acids encodes a coenzyme A (CoA) ligase selected from the group consisting of Nicotiana tabacum 4-coumaryl-CoA ligase, Rhodopseudomonas palstri benzoate-CoA ligase, and Streptomyces coelicolor phenylacetate-CoA ligase.

18. The method of claim 17, wherein the CoA ligase has the amino acid sequence of any one of SEQ ID NOs: 1-3 or an amino acid sequence at least 70% identical to SEQ ID NOs: 1-3 and having CoA ligase activity.

19. The method of of claim 11, wherein one of the plurality of nucleic acids encodes a malonyl-CoA synthethase (MCS), an olivetol synthase (OLS), and an olivetolic acid cyclase (OAC).

20. The method of of claim 19, wherein the MCS has the amino acid sequence of SEQ ID NO: 5 or an amino acid sequence 70% identical to SEQ ID NO: 5 having MCS activity.

21. A prenyltransferase having the amino acid sequence of any one of SEQ ID NOs: 8-11.

22. A nucleic acid encoding a prenyltransferase having the amino acid sequence of any one of SEQ ID NOs: 8-11


SEQUENCE LISTING (12 sequences)

SEQ ID NO: 1 - protein, 542 aa, Nicotiana tabacum
  misc_feature: 4-coumarate-CoA ligase 2
MEKDTKQVDIIFRSKLPDIYIPNHLPLHSYCFENISEFSSRPCLINGANKQIYTYADVELNSRKVAAGLHKQGIQPKDTIMILLPNSPEFVFAFIGASYLGAISTMANPLFTPAEVVKQAKASSAKIIVTQACHVNKVKDYAFENDVKIICIDSAPEGCLHFSVLTQANEHDIPEVEIQPDDVVALPYSSGTTGLPKGVMLTHKGLVTSVAQQVDGENPNLYIHSEDVMLCVLPLFHIYSLNSVLLCGLRVGAAILIMQKFDIVSFLELIQSYKVTIGPFVPPIVLAIAKSPMVDDYDLSSVRTVMSGAAPLGKELEDTVRAKFPNAKLGQGYGMTEAGPVLAMCLAFAKEPFEIKSGACGTVVRNAEMKIVDPKTGNSLPRNQSGEICIRGDQIMKGYLNDPEATARTIDKEGWLYTGDIGYIDDDDELFIVDRLKELIKYKGFQVAPAELEALLLNHPNISDAAVVPMKDEQAGEVPVAFVVRSNGSTITEDEVKDFISKQVIFYKRIKRVFFVDAIPKSPSGKILRKDLRAKLAAGLPN

SEQ ID NO: 2 - protein, 524 aa, Rhodopseudomonas palustri
  misc_feature: benzoate-CoA ligase
MNAAAVTPPPEKFNFAEHLLQTNRVRPDKTAFVDDISSLSFAQLEAQTRQLAAALRAIGVKREERVLLLMLDGTDWPVAFLGAIYAGIVPVAVNTLLTADDYAYMLEHSRAQAVLVSGALHPVLKAALTKSDHEVQRVIVSRPAAPLEPGEVDFAEFVGAHAPLEKPAATQADDPAFWLYSSGSTGRPKGVVHTHANPYWTSELYGRNTLHLREDDVCFSAAKLFFAYGLGNALTFPMTVGATTLLMGERPTPDAVFKRWLGGVGGVKPTVFYGAPTGYAGMLAAPNLPSRDQVALRLASSAGEALPAEIGQRFQRHFGLDIVDGIGSTEMLHIFLSNLPDRVRYGTTGWPVPGYQIELRGDGGGPVADGEPGDLYIHGPSSATMYWGNRAKSRDTFQGGWTKSGDKYVRNDDGSYTYAGRTDDMLKVSGIYVSPFEIEATLVQHPGVLEAAVVGVADEHGLTKPKAYVVPRPGQTLSETELKTFIKDRLAPYKYPRSTVFVAELPKTATGKIQRFKLREGVLG

SEQ ID NO: 3 - protein, 448 aa, Streptomyces coelicolor
  misc_feature: phenylacetate-CoA ligase
MSSEPTTGTAPAPRRGEPLPHDLLDDAERLSREQLRELQLDRLRATLRHAYDNVELYRKKFDAAGVTPDDCRSLADLSRFPFTTKADLRDTYPFGMFAVPMADVRRVHASSGTTGRATVVGYTENDLSMWADVVARSIRAAGGRPGHKVHISYGYGLFTGGLGAHYGAERAGCTVIPASGGMTARQVQIIQDFRPEIIMVTPSYMLTLLDEFERQGVDPRTSSLQVGIFGAEPWTEEMRREIEERMDIHAVDIYGLSEVIGPGVAQECVETKDGLHIWEDHFYPEVVDPLTDAVLPGGEEGEIVFTSLTKEALPVIRYRTRDLTRLLPGTARPAFRRMRKVTGRCDDMIILRGVNVFPTQVEEIVLRTPGVAPHFQMRLTERGRMDHMTVRVEARPDAAPEQRDAAARAIAQGVKDGVGVTVEVEVVEPETLERSLGKIRRVWDQRGA

SEQ ID NO: 4 - protein, 543 aa, Cannabis sativa
  misc_feature: acyl-activating enzyme 3
MEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 5 - protein, 504 aa, Rhizobium trifolii
  misc_feature: malonyl-CoA synthetase
MSNHLFDAMRAAAPGNAPFIRIDNTRTWTYDDAFALSGRIASAMDALGIRPGDRVAVQVEKSAEALILYLACLRSGAVYLPLNTAYTLAELDYFIGDAEPRLVVVASSARAGVETIAKPRGAIVETLDAAGSGSLLDLARDEPADFVDASRSADDLAAILYTSGTTGRSKGAMLTHGNLLSNALTLRDFWRVTAGDRLIHALPIFHTHGLFVATNVTLLAGASMFLLSKFDPEEILSLMPQATMLMGVPTFYVRLLQSPRLDKQAVANIRLFISGSAPLLAETHTEFQARTGHAILERYGMTETNMNTSNPYEGKRIAGTVGFPLPDVTVRVTDPATGLALPPEQTGMIEIKGPNVFKGYWRMPEKTAAEFTADGFFISGDLGKIDRDGYVHIVGRGKDLVISGGYNIYPKEVEGEIDQIEGVVESAVIGVPHPDFGEGVTAVVVRKPGAALDEKAIVSALQDRLARYKQPKRIIFAEDLPRNTMGKVQKNILRQQYADLYTRT

SEQ ID NO: 6 - protein, 385 aa, Cannabis sativa
  misc_feature: olivetol synthase
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 7 - protein, 101 aa, Cannabis sativa
  misc_feature: olivetolic acid cyclase
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 8 - protein, 307 aa, Artificial Sequence
  Streptomyces sp. CL190 NphB V49W, Y288A
MSEAADVERVYAAMEEAAGLLGVACARDKIYPLLSTFQDTLVEGGSVVWFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAAYHITDVQRGLLKAFDSLED

SEQ ID NO: 9 - protein, 307 aa, Artificial Sequence
  Streptomyces sp. CL190 NphB V49W, Y288P
MSEAADVERVYAAMEEAAGLLGVACARDKIYPLLSTFQDTLVEGGSVVWFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAPYHITDVQRGLLKAFDSLED

SEQ ID NO: 10 - protein, 307 aa, Artificial Sequence
  Streptomyces sp. CL190 NphB V49W, Y288A, Q295F
MSEAADVERVYAAMEEAAGLLGVACARDKIYPLLSTFQDTLVEGGSVVWFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAAYHITDVFRGLLKAFDSLED

SEQ ID NO: 11 - protein, 307 aa, Artificial Sequence
  Streptomyces sp. CL190  NphB V49W, Y288P, Q295F
MSEAADVERVYAAMEEAAGLLGVACARDKIYPLLSTFQDTLVEGGSVVWFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAPYHITDVFRGLLKAFDSLED

SEQ ID NO: 12 - protein, 538 aa, Artificial Sequence
  VSP-CBDA fusion
MFSLKALLPLALLLVSANQVAANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH